(19) United States
(12) Patent Application Publication
(10) Pub. No.: US 2026/0110702 A1
(43) Pub. Date: Apr. 23, 2026

(54) METHOD FOR SENSOR CALIBRATION

(71) Applicant: Roche Diagnostics Operations, Inc., Indianapolis, IN (US)

(72) Inventors: Iwo Koenig, Uerzlikon (CH); Milenko Kostadinovic, Luzern (CH); Scott Perala, Urdorf (CH)

(21) Appl. No.: 19/120,457

(22) PCT Filed: Oct. 9, 2023
(86) PCT No.: PCT/EP2023/077850

§ 371 Date: Apr. 11, 2025

(30) Foreign Application Priority Data
Oct. 11, 2022 (EP) 22200811.2

(51) Int. Cl. G01N 33/96; G01N 35/08

(52) U.S. Cl. CPC G01N 33/96; G01N 35/08; G01N 35/085; G01N 2496/70; G01N 2496/80

(57) ABSTRACT

Please add the following Abstract on a separate sheet after the claims section of the subject application.

An in-vitro diagnostic (IVD) analyzer 200 comprising at least one sensor 212 located in a flow-through sensor path 211 of detecting unit and requiring at least one oxygenated calibration fluid 221′, 222′ for calibration is herein disclosed. The IVD analyzer 200 further comprises a fluid-supply unit 220 comprising at least one deoxygenated calibration fluid 221, 222, a fluid-selection valve 230 and at least one oxygenation tubing 215, 216 having two ends connected to the fluid-selection valve 230 as a loop, wherein the oxygenation tubing 215, 216 comprises oxygen-permeable walls, and wherein the IVD analyzer 200 further comprises a pump 240 and a controller 250 configured to control the pump 240 and the fluid-selection valve 230 for transporting deoxygenated calibration fluid 221, 222 into the oxygenation tubing 215, 216, to wait a predetermined time required for oxygenation of the deoxygenated calibration fluid 221, 222 via oxygen uptake from ambient air through the tubing walls, and to transport the thereby obtained oxygenated calibration fluid 221′, 222′ into the sensor path 211 for calibration of the at least one sensor 212. A respective automatic method of calibrating at least one sensor 212 is herein also disclosed.

### FIELD OF THE INVENTION

The present invention refers to an automated method of calibrating a sensor located in a flow-through sensor path and requiring at least one oxygenated calibration fluid for calibration, as well as to a respective in-vitro diagnostic analyzer.

### BACKGROUND

In medicine, doctor's diagnosis and patient treatment often relies on the measurement of patient sample parameters carried out by in-vitro diagnostic analyzers. It is important that the analyzers perform correctly by providing precise and reliable measurements. Thus, it is a general requirement for in-vitro diagnostic analyzers to implement a set of procedures to ensure measurement performance. One of these procedures is calibration. In most cases calibration is performed using standard solutions, with known concentrations or parameters. In this way it is possible to correlate a measured signal to a quantitative result. Calibration should be performed more or less frequently depending on the system and other variable factors which may affect performance.

In blood gas and electrolyte testing, parameters are determined from a patient's sample, like the partial pressure of blood gases (pO₂, pCO₂), oxygen saturation (SO₂), pH value, electrolyte concentrations (e.g. Na^{|}, K^{|}, Mg₂^{|}, Ca₂^{|}, Li^{|}, Cl) , bicarbonate values (HCO₃⁻) , the concentration of metabolites (e.g. glucose, lactate, urea, creatinine), values for hemoglobin and hemoglobin derivatives (e.g. tHb, O₂Hb, HHb, COHb, MetHb, SulfHb), bilirubin values, and hematocrit.

Typically, the parameters are determined by conductivity, electrochemical and/or optical measuring principles. Sensors based on these measuring principles and configured to measure such sample parameters may be combined, e.g. arranged sequentially in one or more flow-through sensor paths. This allows for simultaneous and/or sequential determination of a plurality of parameters from one single sample in one single test run.

Some metabolite sensors, e.g. Glucose and Lactate sensors, require the presence of oxygen to perform measurements. In order to calibrate these sensors, calibration fluids containing lactate and/or glucose respectively and also oxygen at known levels are therefore required. Glucose/Lactate and oxygen however cannot be stored together as a ready to use calibration fluid as they react with each other, thereby changing their respective contents over storage time. Therefore Glucose/Lactate calibration fluids are typically stored without oxygen, thus requiring oxygen to be added just before sensor calibration in a process called tonometry. This process typically comprises drawing a predetermined amount of deoxygenated calibration solution into a fluidic line made of a material permeable to oxygen from ambient air, e.g. made of silicone, and waiting a predetermined time for oxygen diffusion through the walls of the fluidic line into the calibration fluid until a required level of oxygenation is reached before drawing this freshly oxygenated (tonometered) calibration fluid into the sensor path and calibrating the sensor.

Since tonometry is only one of the steps required for executing a calibration procedure and different fluids are sequentially transported through the same fluidic line via the same pump, the waiting step for diffusion to occur can unduly extend the calibration time. Also, as several calibration fluids requiring tonometry may be required, in order for example to execute multi-point calibrations, the tonometry process may be even more time-consuming.

### GENERAL DESCRIPTION

It is against the above background that aspects of the present disclosure provide certain unobvious advantages and advancements over the prior art. In particular, a new automated method of calibrating a sensor located in a flow-through sensor path of a detecting unit of an in-vitro diagnostic (IVD)) analyzer involving a reaction with oxygen in a sample in order to determine a sample parameter and requiring at least one oxygenated calibration fluid with a certain level of oxygenation for calibration is herein disclosed that enables tonometry of deoxygenated calibration fluids without unnecessarily extending the calibration time and/or by enabling parallel steps in the meantime, by preventing blocking the use of the fluidic system and of the pump while waiting for tonometry.

In particular, the method comprises controlling by a controller a pump and a fluid-selection valve, including transporting deoxygenated calibration fluid from a fluid-supply unit into an oxygenation tubing having two ends connected to a fluid-selection valve as a loop, the oxygenation tubing comprising oxygen-permeable walls, the fluid-selection valve comprising one or more fluid input ports for selecting at least one fluid at a time, and a common outlet port fluidically connected or connectable via a fluidic line to the sensor path. The method further comprises waiting a predetermined time required for oxygenation of the deoxygenated calibration fluid via oxygen uptake from ambient air through the tubing walls until the required level of oxygenation is obtained, thereby obtaining an oxygenated calibration fluid, transporting the thereby obtained oxygenated calibration fluid into the sensor path and calibrating the at least one sensor.

An in-vitro diagnostic analyzer configured to execute said automated method and presenting the same advantages is herein also disclosed.

The term “in-vitro diagnostic analyzer” or “IVD analyzer” as used herein refers to an automated or semi-automated analytical apparatus configured to examine samples in vitro in order to provide information for screening, diagnosis or treatment monitoring purposes. The IVD analyzer is designed and configured according to the medical area of application, the parameters to be determined and corresponding laboratory workflows. For example, in a point-of-care testing environment, IVD analyzers can vary from handheld devices with low throughput, short turn-around time and limited number of measurable parameters to compact benchtop instruments with higher throughput and higher number of measurable parameters. Such IVD analyzers are designed to detect certain types of parameters, e.g. gases, electrolytes, metabolites, clinical chemistry analytes, immunochemistry analytes, coagulation parameters, hematology parameters, etc. Depending on the parameters of interest, a variety of different analytical methods and different detection technologies can be applied. For example, in the field of blood gas and electrolyte testing, electrochemical measuring principles and/or conductivity measuring principles and/or optical detection methods are used. An IVD analyzer typically comprises a plurality of functional units, each dedicated to a specific task and cooperating with each other in order to enable automated sample processing and analysis. Such functional units may include e.g. a sample input interface for receiving a sample, a fluidic system, an analytical measurement unit or detecting unit, a fluid-supply unit, and the like. One or more functional units may be integrated into a larger unit or module in order to simplify the operation of the IVD analyzer.

In particular, the in-vitro diagnostic (IVD) analyzer of the present disclosure comprises at least one sensor located in a flow-through sensor path of a detecting unit involving a reaction with oxygen in a sample in order to determine a sample parameter and requiring at least one oxygenated calibration fluid with a certain level of oxygenation for calibration. The IVD analyzer further comprises a fluid-supply unit comprising at least one deoxygenated calibration fluid, a fluid-selection valve comprising one or more fluid input ports for selecting at least one fluid at a time, a common outlet port fluidically connected or connectable via a fluidic line to the sensor path. The IVD analyzer further comprises at least one oxygenation tubing having two ends each connected to the fluid-selection valve, thereby forming a loop, the oxygenation tubing comprising oxygen-permeable walls. The IVD analyzer further comprises a pump and a controller configured to control the pump and the fluid-selection valve for transporting the at least one deoxygenated calibration fluid from the fluid-supply unit into the oxygenation tubing, to wait a predetermined time required for oxygenation of the deoxygenated calibration fluid via oxygen uptake from ambient air through the tubing walls until the required level of oxygenation is obtained, and to transport the thereby obtained oxygenated calibration fluid into the sensor path for calibration of the at least one sensor.

A “detecting unit” according to the present disclosure is an analytical measurement unit of the IVD analyzer comprising at least one flow-through sensor path. A “flow-through sensor path” is a fluidic conduit that may comprise one or more sensors that a sample flowing through the sensor path comes in contact with, e.g. arranged sequentially along the path, e.g. a sensor for each different parameter/analyte to be detected, and may be embodied in a replaceable cartridge-like structure comprising a plurality of sensors, possibly distributed across a plurality of sensor paths. In alternative, the IVD analyzer may comprise a plurality of detecting units, each having a sensor path comprising a sensor dedicated to one parameter/analyte, and which may also be replaceable or not. A sample may thus flow into the one or more sensor paths and different parameters/analytes may be determined by respective sensors. The detecting unit may optionally comprise also a flow-through optical measurement unit. The flow-through sensor path may be an integrated part of the fluidic system of the IVD analyzer or part of a separate component such as for example a sensor cartridge fluidically connected to the fluidic system of the IVD analyzer, in a way that the at least one fluidic line and the al least one sensor path are fluidically connected.

The term “sensor” is herein generically used to indicate a detector configured to detect sample parameters by generating a correlated signal output that can be quantified and digitized. The sensor can be e.g. a biosensor, a chemical sensor or a physical sensor and is typically a part of a functional unit of an IVD analyzer, e.g. an analytical measurement unit or detecting unit. The sensor can be selective or specific with respect to one sample parameter of interest or can be configured to detect and quantify a plurality of different sample parameters of interest. Depending on the type of sensor, a sensor can comprise a plurality of sensory elements. The term “sensory element” therefore refers to a part of a sensor (e.g. to a working electrode, a reference electrode, a counter electrode) that in combination with one or more other sensory elements forms a fully functional sensor.

According to certain aspects the detecting unit comprises any one or more of a pO₂ sensor, a pCO₂ sensor, a pH sensor, one or more ion selective electrode (ISE) sensors for determining electrolyte values such Na⁺, K⁺, Ca₂⁺ and Cl⁻, one or more metabolite sensors for determining parameters such as lactate and glucose. The sensors may be e.g. respectively based on the amperometric, potentiometric or conductometric principle.

For example, a pO₂ sensor typically functions according to the Clark measurement principle. This means that oxygen diffuses through a membrane to a gold multi-wire system with negative electric potential inside of the sensor. The oxygen is reduced here, which generates an electric current that is proportional to the oxygen contained in the sample. This current is measured amperometrically.

A pCO₂ sensor typically is a Severinghouse-type sensor. This means that CO₂ diffuses through a membrane similar to the oxygen sensor. In the sensor, the CO₂ concentration changes and causes a correlated change of the pH value of an internal buffer system, which is measured potentiometrically.

A pH sensor typically comprises a pH-sensitive membrane. Depending on the pH value of the test sample, electric potential is generated at the boundary layer between the membrane and the sample. This potential can be measured potentiometrically by a reference sensor.

Na⁺, K⁺, Ca₂₊ and Cl⁻ ISE sensors typically work according to the potentiometric measuring principle. They differ only by different membrane materials that enable sensitivity for the respective electrolytes.

The glucose sensor typically makes use of glucose oxidase enzyme, by which glucose is oxidized into gluconolactone with oxygen available in the sample. The H₂O₂ that is formed in this process is determined amperometrically by a manganese dioxide/carbon electrode.

The Lactate sensor typically makes use of lactate oxidase enzyme, by which lactate is oxidized into pyruvate with oxygen available in the sample. The H₂O₂ that is formed in this process is determined amperometrically in a manner similar to the glucose sensor.

According to an embodiment, the at least one sensor is a metabolite sensor including at least one of a glucose sensor and a lactate sensor, or any other biosensor involving a reaction with oxygen.

A “fluidic line” is part of a larger fluidic system that can include one or more hollow conduits such as a tubing, a channel, a chamber or combinations thereof, comprising one or more parts, suitable for the passage of fluids in at least a liquid tight manner, and which can have any shape and size, but which is typically optimized to minimize internal volumes and dead volumes. The parts may be flexible, rigid or elastic or combinations thereof. The one or more fluidic lines can be connected or connectable at least in part to each other, e.g. via fluidic connection and/or valves.

The term “fluid-selection valve” refers to a flow-regulating device to control, redirect, restrict or stop flow and, in particular, to a switching or rotary valve, that is a multi-port valve that enables to select fluidic connections. This is typically achieved by moving one or more valve conduits to switch communication between different elements. Elements may be fluidically connected via further conduits, like pipes, tubes, capillaries, microfluidic channels and the like. For example, the valve may be integrated into a manifold comprising a channel and a respective input port for each fluid reservoir, where upon switching, e.g. by rotation, of the valve a fluidic connection is established between one fluidic reservoir at a time and e.g. a common outlet port connected or connectable to the fluidic line leading to the flow-through sensor path. Alternatively, the manifold may comprise an input port for each fluid reservoir all leading to a common channel and to a common fluid input port of the fluid-selection valve, in which case an individual on/off valve may be arranged in correspondence to each fluid reservoir or fluid input port for allowing selected fluids from selected fluid reservoirs to flow into the common channel and to the fluid selection valve. The valve may comprise other input ports for other fluids like for ambient air and/or for samples.

The IVD analyzer also comprises at least one pump, e.g. a peristaltic pump, syringe pump, membrane pump or any other suitable pump, for transporting e.g. samples from sample containers, other fluids from the fluid-supply unit, or ambient air through the at least one fluidic line and through the detecting unit. In particular, the pump is used for transporting a deoxygenated calibration fluid from the fluid-supply unit into the oxygenation tubing via the fluid-selection valve and for transporting the obtained oxygenated calibration from the oxygenation tubing into the sensor path. The pump is typically positioned downstream of the detecting unit but it may be located at any other position and may be connected to the fluidic system via further elements such as valves and switches. Also, the pumping direction can be invertible.

A “fluid-supply unit” as used herein is a module or component of the IVD analyzer comprising one or more fluid reservoirs, and possibly also one or more waste containers where fluids circulated through the fluidic system may be disposed of at the end of the process. The term “fluid” may refer to either a gas or liquid or mixtures thereof. A fluid may be for example a sample, a reagent, a reference fluid such as a quality control fluid or a calibration fluid, a cleaning fluid, a wetting fluid, air or other gas.

Samples are typically entered into the fluidic system via a sample input interface, different from the fluid-supply unit, that is another module or component of an in-vitro diagnostic analyzer typically arranged at a position conveniently accessible by an operator and configured to transfer a sample from a sample container brought up by the operator into the in-vitro diagnostic analyzer. The sample input interface may e.g. comprise a sample input port comprising an outer input-port side configured for coupling, attaching, connecting, sitting, introducing or plugging-in a sample container, e.g. of the capillary-type or syringe-type, and an inner input-port side coupled to or for coupling to one end of a sample-input conduit, the sample input conduit being for example fluidically connected or connectable at the other end to the detecting unit/sensor path directly or via the fluid-selection valve. According to one embodiment the sample-input conduit is the same fluidic line leading from the fluid-selection valve to the sensor path and configured to alternately connect with the same end to the inner input port side and to the fluid-selection valve, e.g. directly to the outlet port of the fluid-selection valve, or to a further port connected to the outlet port of the fluid-selection valve via a further conduit, in order to alternately draw a sample from the sample input port and a fluid other than a sample through the fluid-selection valve, whereas the other end is connected to the detecting unit/sensor path. According to an embodiment the fluidic line, if acting also as sample input-conduit, may be embodied at least in part as a rigid aspiration needle configured to be coupled to the inner input-port side of the sample input port and to the outlet port of the fluid selection-valve or extension thereof. According to an embodiment ambient air can also be aspirated via the sample input port directly into the fluidic line or via a dedicated air port of the fluid selection valve.

A “calibration fluid” is a reference or standard solution, typically provided in the fluid-supply unit, that contains known values of one or more calibration substances used for calibration and that is measured under the same conditions as a biological sample. A calibration substance can be an analyte identical to an analyte of interest potentially present in a biological sample and being detected by a particular sensor, the concentration of which is however known, or that generates by reaction an analyte identical to an analyte of interest, the concentration of which is known, or it can be any other equivalent substance of known concentration, which mimics a sample parameter of interest or that can be otherwise correlated to a certain parameter of interest, e.g. a dye that behaves optically similarly to an analyte of interest. Typically, one or two calibration fluids are used for a one-point or two-point calibration respectively, when the sensor responds linearly to analyte concentrations. Three or more calibration fluids may be used if the calibration curve is non-linear. In particular, calibration fluids can be provided in different levels that correspond to different concentration ranges of the calibration substances.

In particular, at least one deoxygenated calibration fluid is provided in the fluid-supply unit according to the present disclosure. The term “deoxygenated” refers to a level of oxygen that is either zero or sufficiently low that the content of any other calibration substance in the calibration fluid does not significantly change over the storage time as a result of reaction with the oxygen contained therein. In other words the level or partial pressure of oxygen in the calibration fluid is such that the shelf life of the calibration fluid is not affected by its oxygen level. Deoxygenation may be obtained upon manufacturing by e.g. degassing the solvent used for the calibration fluid, or replacing oxygen with another inert gas and in general by executing the manufacturing process in a deoxygenated environment so that oxygen uptake from air is prevented before the calibration fluid is sealed in a liquid and gas tight fluid reservoir. In order to maintain the level of oxygenation at low or near zero level during storage, a chemical oxygen scavenger may be used in the deoxygenated calibration fluid, e.g. in order to interact with and neutralize any oxygen eventually permeated into the fluid reservoir during storage, but whose reaction kinetics is slow enough that it does not interfere with the process of oxygenation in the fluidic line.

Oxygenation of the deoxygenated calibration fluid occurs in the oxygenation tubing just in the amount needed and just before use for calibration. In particular, the “oxygenation tubing” is a fluidic conduit, which unlike other parts of the fluidic system, which in order to avoid affecting sample parameters during transport are both liquid tight and gas tight, it is only liquid-tight and is therefore permeable at least lo oxygen from ambient air. The oxygenation tubing therefore comprises oxygen-permeable walls, i.e. made of a material permeable to oxygen, e.g. made of silicone.

According to an embodiment, the IVD analyzer comprises an oxygenation tubing for each different deoxygenated calibration fluid to be oxygenated.

According to an embodiment, the method therefore comprises transporting different deoxygenated calibration fluids to be oxygenated into respective oxygenation tubings.

The term “oxygenated” with respect to the calibration fluid refers to a level of oxygenation or partial pressure of oxygen, obtained after transporting deoxygenated calibration fluid into the oxygenation lubing and waiting a predetermined time for oxygen uptake from ambient air through the walls of the oxygenation tubing. The predetermined time is the time required to obtain a level of near saturation or equilibrium corresponding to the required level of oxygenation or partial pressure of oxygen that is most suitable and/or sufficient for calibrating the at least one sensor, as prolonging this waiting time would negligibly contribute to further increase the oxygenation level and the process would be unnecessarily prolonged. The partial pressure of oxygen in the obtained oxygenated calibration fluid is typically about 80-90% of the partial pressure of oxygen in ambient air, that is about 110-170 mmHg, depending e.g. on altitude.

The difference between a deoxygenated calibration fluid and an oxygenated calibration fluid according to the present disclosure is given only by the different level of oxygenation respectively.

The term “controller” as used herein may include any physical or virtual processing device and in particular a programmable logic controller running a computer-readable program provided with instructions to perform operations in accordance with an operation plan and in particular in accordance with the process of calibrating a sensor as herein disclosed. This may include a processor, a controller, a central processing unit (CPU), a microprocessor, a microcontroller, a reduced instruction circuit (RISC), an application-specific integrated circuit (ASIC), a logic circuit, or any other circuit or processor configured to execute one or more of the functions/methods described herein. In particular, the controller is configured to control the pump and the fluid-selection valve for transporting deoxygenated calibration fluid from the fluid-supply unit into the oxygenation tubing, to wait a predetermined time required for oxygenation of the deoxygenated calibration fluid via oxygen uptake from ambient air through the tubing walls, and to transport the thereby obtained oxygenated calibration fluid into the sensor path for calibration of the at least one sensor.

According to an embodiment, the controller is further configured to control the pump and the fluid-selection valve to transport any other fluid from the fluid-supply unit into the sensor path while the al least one oxygenation tubing is fluidically isolated and the deoxygenated calibration fluid is being oxygenated.

According to an embodiment, the method therefore comprises controlling the pump and the fluid-selection valve to transport any other fluid from the fluid-supply unit into the sensor path while the at least one oxygenation tubing is fluidically isolated and the deoxygenated calibration fluid is being oxygenated.

According to an embodiment, the controller is further configured to control the pump and the fluid-selection valve to transport the oxygenated calibration fluid out of the oxygenation tubing into the sensor path while introducing fresh deoxygenated fluid into the oxygenation tubing to be oxygenated. According to an embodiment, the method therefore comprises controlling the pump and the fluid-selection valve to transport the oxygenated calibration fluid out of the oxygenation tubing into the sensor path while introducing fresh deoxygenated fluid into the oxygenation tubing to be oxygenated. Other and further objects, features and advantages will appear from the following description of exemplary aspects and accompanying drawings, which serve to explain the principles more in detail.

### BRIEF DESCRIPTION OF THE DRAWINGS

FIG. 1A shows schematically an in-vitro diagnostic analyzer comprising a flow-through sensor path and a first step of an automated method of calibrating a sensor located in the flow-through sensor path.

FIG. 1B shows schematically the same in-vitro diagnostic analyzer of FIG. 1A and a second step of the same automated method.

FIG. 1C shows schematically the same in-vitro diagnostic analyzer of FIG. 1A-1B and a third step of the same automated method.

FIG. 1D shows schematically the same in-vitro diagnostic analyzer of FIG. 1A-1C and a fourth step of the same automated method.

FIG. 1E shows schematically the same in-vitro diagnostic analyzer of FIG. 1A-1D and a fifth step of the same automated method.

FIG. 2 shows schematically the same in-vitro diagnostic analyzer of FIG. 1A-1E and a step of transporting a sample for analysis after sensor calibration.

FIG. 3 shows schematically the same in-vitro diagnostic analyzer of FIG. 1A-1E and a variant of the step shown in FIG. 1E.

FIG. 4 shows more in detail and in cross section parts of a fluid-selection valve integrated into a manifold and an oxygenation tubing, during the step shown in FIG. 1A.

FIG. 5 shows similar details as in FIG. 4 during the step shown in FIG. 3.

FIG. 6 looks like FIG. 4 but is in connection to the step shown in FIG. 1D.

FIG. 7 shows the progressive level of oxygenation achieved with different predetermined times for oxygen uptake in the oxygenation tubing.

Skilled artisans appreciate that elements in the figures are illustrated for simplicity and clarity and have not necessarily been drawn to scale. For example, the dimensions of some of the elements in the figures may be exaggerated relative to other elements whereas other elements may have been left out or represented in a reduced number in order to enhance clarity and improve understanding of the aspects of the present disclosure.

### DETAILED DESCRIPTION

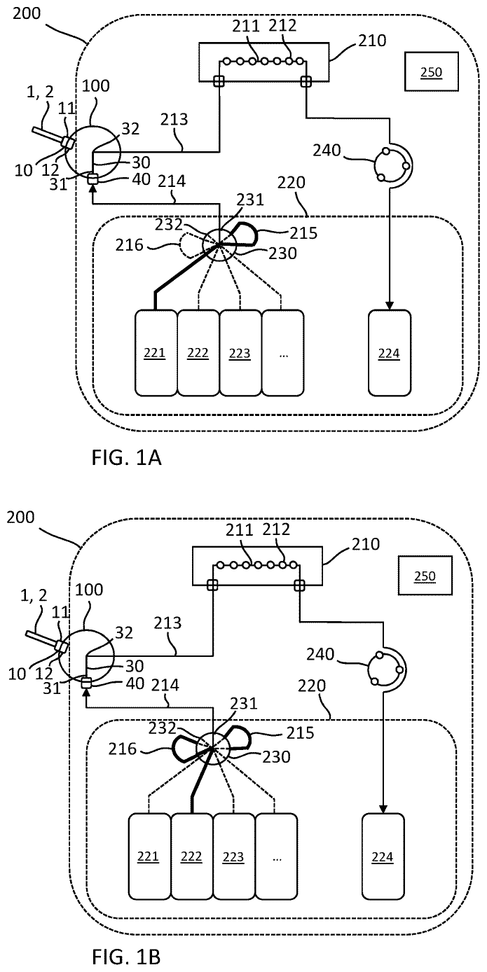

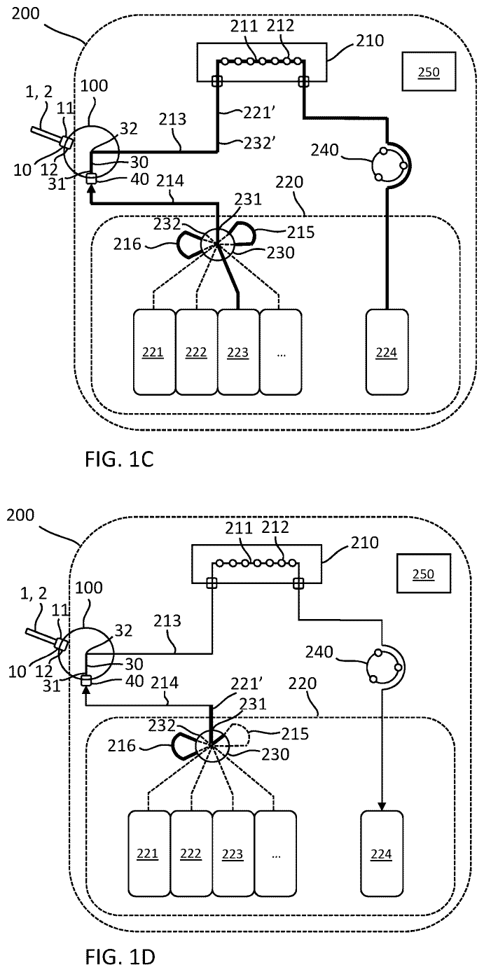

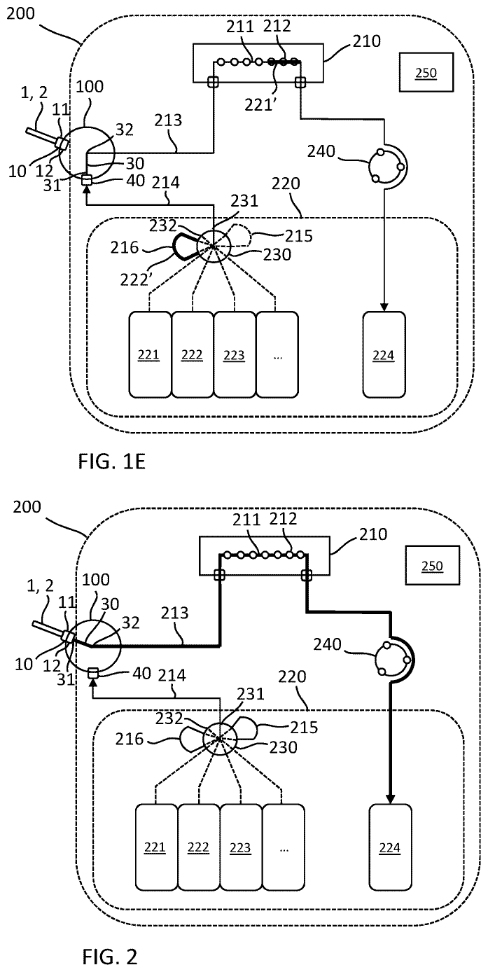

FIG. 1A to FIG. 1E taken together show schematically an example of in-vitro diagnostic analyzer 200 comprising a flow-through sensor path 211 according to the present disclosure as well as an automated method of calibrating a sensor 212 located in the flow-through sensor path 211. In particular, the in-vitro diagnostic analyzer 200 comprises a detecting unit 210 comprising a flow-through sensor path 211, the flow-through sensor path 211 comprising at least one sensor 212 involving a reaction with oxygen in a sample in order to determine a sample parameter and requiring at least one oxygenated calibration fluid with a certain level of oxygenation for calibration. According to an embodiment, the at least one sensor 212 is a metabolite sensor including at least one of a glucose sensor and a lactate sensor.

The IVD analyzer 200 further comprises a fluid-supply unit 220 comprising at least one deoxygenated calibration fluid 221, 222 among other fluids 223, a fluid-selection valve 230 for selecting at least one fluid 221, 222, 223 at a time, and a fluidic line 213 comprised between the valve 230 and the sensor path 211. The IVD analyzer 200 further comprises a sample input interface 100, comprising a sample input port 10 comprising an outer input-port side 11 configured for plugging-in an open end of a sample container 1 and an inner input-port side 12. The sample container 1 is in this example is a capillary-like sample container. The sample input interface 100 further comprises an aspiration needle 30 comprising an upstream end 31 and a downstream end 32. The downstream end 32 of the aspiration needle 30 is fluidically connected to the sensor path 211 via the fluidic line 213 whereas the upstream end 31 is configured to alternately couple to the inner input-port side 12 in order to aspirate a sample from a sample container 1 plugged in the outer input-port side 11 and to a fluid supply unit port 10 fluidically connected to a common outlet port 231 of the fluid-selection valve 230 via a further conduit 214. The fluidic line 213 may be however directly connected to the outlet port 231 of the fluid-selection valve 230, whereas samples may be introduced via a different fluidic line separately connected to the fluid-selection valve 230, for example. The fluid-selection valve comprises also an air port 232. The IVD analyzer 200 further comprises in this example two oxygenation tubings 215, 216 each having two ends connected to the fluid-selection valve 230 as a loop, the oxygenation tubings comprising oxygen-permeable walls.

The IVD analyzer 200 further comprises a pump 240, such as a peristaltic pump, located downstream of the sensor path 211 and also a waste container 224, located in the fluid-supply unit 220, where fluids circulated through the fluidic line 213 and sensor path 211 may be disposed of.

The IVD analyzer 200 further comprises a controller 250 configured to automatically execute any of the herein disclosed method steps.

In particular, FIG. 1A shows schematically, the position of the fluid being indicated by bold lines, a first step of the automated method of calibrating the at least one sensor 212 located in the flow-through sensor path 211 of a detecting unit 210 of an in-vitro diagnostic (IVD) analyzer 200 involving a reaction with oxygen in a sample in order to determine a sample parameter and requiring at least one oxygenated calibration fluid with a certain level of oxygenation for calibration. The method comprises controlling by the controller 250 the pump 240 and the fluid-selection valve 230, including transporting a first deoxygenated calibration fluid 221 from the fluid-supply unit 220 into a first oxygenation tubing 215.

The method continues, as shown in FIG. 1B, by waiting a predetermined time required for oxygenation of the deoxygenated calibration fluid 221via oxygen uptake from ambient air through the walls of the oxygenation tubing 215 until the required level of oxygenation is obtained, thereby obtaining a first oxygenated calibration fluid. The method further comprises transporting a second deoxygenated calibration fluid 222 to be oxygenated into a second respective oxygenation tubing 216, while the first oxygenation tubing 215 is fluidically isolated and the first deoxygenated calibration fluid 221 is being oxygenated.

As shown in FIG. 1C, the method further comprises controlling the pump 240 and the fluid-selection valve 230 to transport any other fluid 223 from the fluid-supply unit 220 into the sensor path 211 while the oxygenation tubings 215, 216 are fluidically isolated and the calibration fluids are being oxygenated.

As shown in FIG. 1D and FIG. 1E, the method further comprises transporting the obtained first oxygenated calibration fluid 221′ from the oxygenation tubing 215 into the sensor path 211 and calibrating the at least one sensor 212, while the second deoxygenated calibration fluid 222 continues the oxygenation step. Once the oxygenation step for the second deoxygenated calibration fluid 222 is completed, the method comprises transporting also the second obtained second oxygenated calibration fluid 222′ from the second oxygenation tubing 216 into the sensor path 211 and calibrating the at least one sensor 212 (not shown).

FIG. 2 shows schematically the same in-vitro diagnostic analyzer 200 of FIG. 1A-1E and a step of transporting a sample 2 from the sample container 1 plugged in the outer input-port side 11 of the sample input port 10, while the upstream end 31 of the aspiration needle 30 is coupled to the inner input-port side 12 of the sample input port 10, for analysis of the sample 2 by the sensor 212 after calibration according to the method of FIG. 1A-1E. After calibration and before transporting a sample, an intermediate cleaning step with another fluid may take place (not shown). The controller 250 is in this case also configured to control the aspiration needle 30 to alternately couple to the inner input-port side 12 in order to aspirate a sample from a sample container 1 plugged in the outer input-port side 11 and to the fluid-supply unit port 40 for performing the other steps. The step of transporting a sample and analyzing the sample may also be executed while the calibration fluid is in the oxygenation tubing and the oxygenation step is ongoing.

In general, the method of FIG. 1A-1E enables tonometry of deoxygenated calibration fluids without unnecessarily extending the calibration time and/or enables parallel steps in the meantime, by preventing blocking the use of the fluidic system and of the pump while waiting for oxygenation.

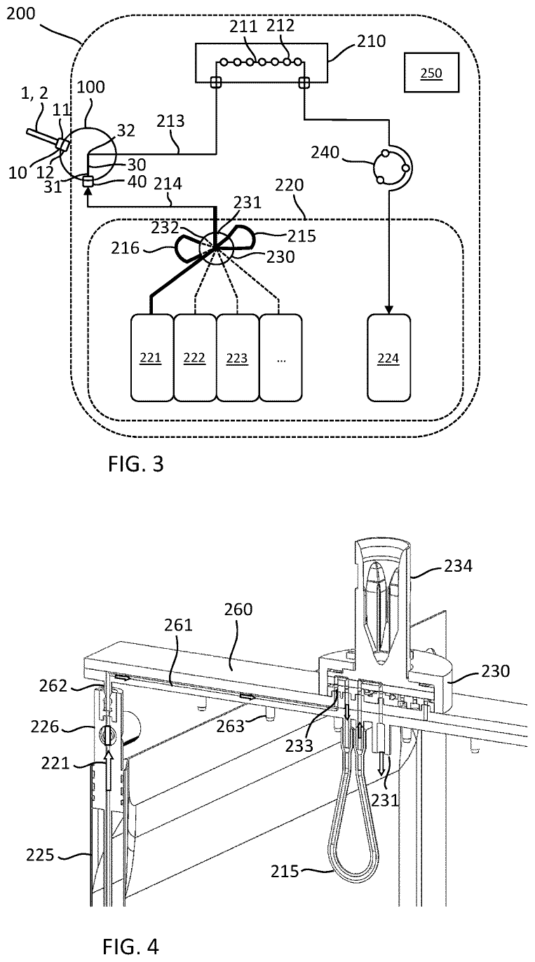

FIG. 3 shows schematically the same in-vitro diagnostic analyzer of FIG. 1A-1E and a variant of the step shown in FIG. 1E. This variant of the method comprises controlling the pump 240 and the fluid-selection valve 230 to transport the oxygenated calibration fluid 221′ out of the oxygenation tubing 215 into the sensor path 211 while introducing fresh deoxygenated fluid 221 into the oxygenation tubing 215 to be oxygenated.

FIG. 4 shows more in detail, a perspective and cross-sectional view of parts of an embodiment comprising a fluid selection valve 230 integrated into a manifold 260, of an oxygenation tubing 215 connected to the manifold 260 as a loop and of a fluid reservoir 225 with an on/off valve 226 also connected to the manifold 260. In particular, the manifold 260 comprises a channel 261 and a respective input port 262, 263 for each fluid reservoir 225 (only one fluid reservoir shown for simplicity). The input ports 262, 263 all lead to the same and common channel 261 and to a common fluid input port 233 of the fluid-selection valve 230. Each fluid reservoir 225 comprises an individual on/off valve 226 by which the fluid reservoir 225 is connected to the respective input port 262 of the manifold 260, in order to allow selected fluids 221 from selected fluid reservoirs 225 to flow into the common channel 261 and to the fluid-selection valve 230. FIG. 4 shows schematically, by means of arrows, the same step shown also in FIG. 1A comprising transporting a deoxygenated calibration fluid 221 from a fluid reservoir 225 into an oxygenation tubing 215 (only one oxygenation tubing shown for simplicity). In particular, the fluid-selection valve 230 is in a switch position, obtained by rotation of an actuation member 234, that enables the calibration fluid to flow via the fluid input port 233 into the fluid selection valve 230 and via the fluid-selection valve 230 through the oxygenation tubing 215 and again via the fluid-selection valve 230 out via the outlet port 231. The on/off valve 226 is in the on status while all other on/off valves (not shown) of the other fluid reservoirs (not shown) are in the off status.

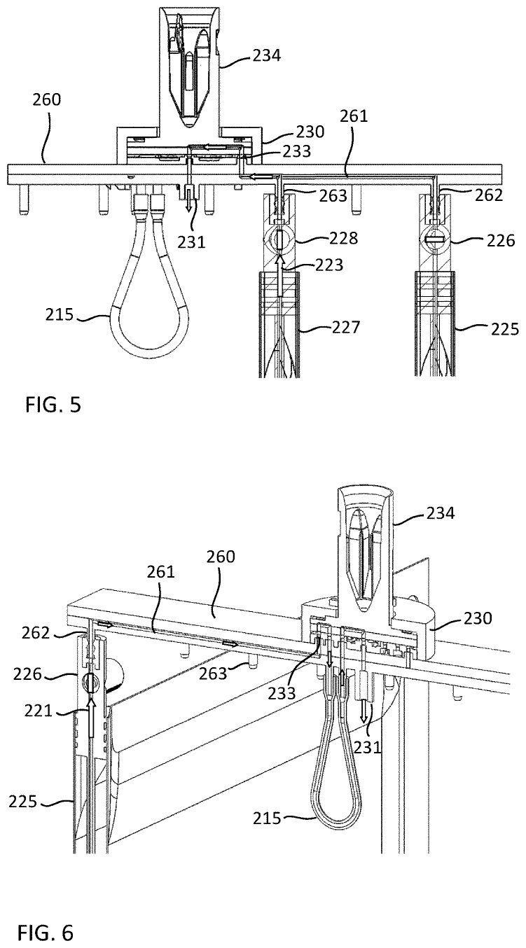

FIG. 5 shows a front and partly cross-sectional view with details and parts similar to that shown in FIG. 4. In particular, FIG. 5 additionally shows a second fluid reservoir 227 containing fluid 223, in this example different from a calibration fluid, connected to the input port 263 of the manifold 260 via the on/off valve 228. The on/off valve 228 is in the on status while the on/off valve 226 and all other on/off valves (not shown) of other fluid reservoirs (not shown) are in the off status. The fluid-selection valve 230 is in a switch position, obtained by rotation of the actuation member 234, that enables the different fluid 223 to flow via the manifold channel 261 and the fluid input port 233 into the fluid selection valve 230 and via the fluid-selection valve 230 to the outlet port 231 leading to the sensor path (not shown in FIG. 5), while the oxygenation tubing 215, with the calibration fluid inside being oxygenated, is fluidically isolated. The oxygenation tubing 215 is made of a material permeable to oxygen from ambient air, e.g. made of silicone. Thus, FIG. 5 also shows, schematically by means of arrows, the same step shown also in FIG. 1C of transporting any other fluid 223 from the fluid-supply unit into the sensor path while the oxygenation tubing 215 is fluidically isolated and the deoxygenated calibration fluid is being oxygenated.

FIG. 6 is nearly identical to FIG. 1 but schematically showing, also by means of arrows, the same step shown in FIG. 3, comprising transporting the oxygenated calibration fluid 221′ out of the oxygenation tubing 215 into the sensor path (not shown in FIG. 6) via the outlet port 231 while introducing fresh deoxygenated fluid 221 into the oxygenation tubing 215 to be oxygenated.

With continued reference to FIG. 4-6 the controller (not shown) is configured to control also the on/off valves 226, 228 besides the fluid-selection valve 230 and the pump 240 such as to select a fluid at a time.

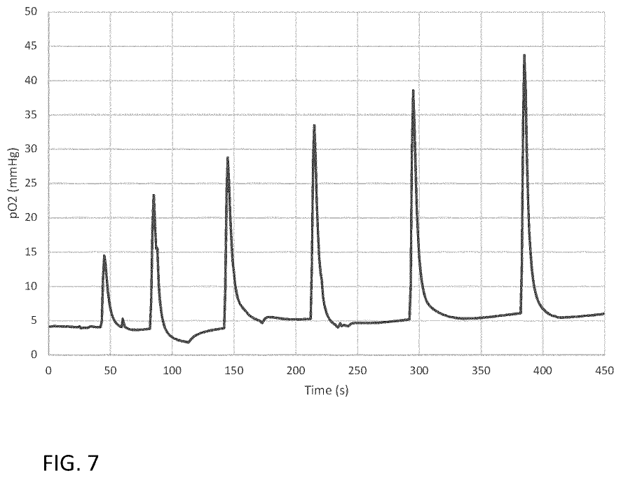

FIG. 7 is a diagram showing the progressive level of oxygenation of a calibration fluid achieved over time in an oxygenation tubing as used in the above examples, by measuring the partial pressure of oxygen pO₂ in mmHg at different time points, e.g. by repeating the process with progressively longer waiting times and measuring the respective pO₂ of the partially oxygenated calibration fluid in the sensor path. It can be seen that in order to obtain a sufficient level oxygenation, that is typically about 80-90% of the partial pressure of oxygen in ambient air, that is about 110-170 mmHg, depending e.g. on altitude of it may be required to wait several minutes, beyond the time shown in the diagram. Although not explicitly illustrated in the above examples, it is clear that several other steps may be executed during this time, thanks to the fact that fluidic system and the pump remain available for use during this time.

Modifications and variations of the disclosed aspects are also certainly possible in light of the above description. It is therefore to be understood, that within the scope of the appended claims, the invention may be practiced otherwise than as specifically devised in the above examples.

Particularly, it is to be understood that at least some of the drawings or parts are only schematic and provided as way of example only. Also the relationship between elements may be other than the one shown, whereas parts not relevant for the purpose of this disclosure have been omitted.

Also, reference throughout the preceding specification to “one example” or “an example”, means that a particular feature, structure or characteristic described in connection with the aspect or example is included in at least one aspect. Thus, appearances of the phrases “one example” or “an example”, in various places throughout this specification are not necessarily all referring to the same example.

Furthermore, the particular features, structures, or characteristics may be combined in any suitable combinations and/or sub-combinations in one or more aspects or examples.

1. An in-vitro diagnostic (IVD) analyzer comprising at least one sensor located in a flow-through sensor path of a detecting unit involving a reaction with oxygen in a sample in order to determine a sample parameter and requiring at least one oxygenated calibration fluid with a certain level of oxygenation for calibration, the IVD analyzer further comprising a fluid-supply unit comprising at least one deoxygenated calibration fluid, a fluid-selection valve comprising one or more fluid input ports for selecting at least one fluid at a time, a common outlet port fluidically connected or connectable via a fluidic line to the sensor path, wherein the IVD analyzer further comprises at least one oxygenation tubing having two ends connected to the fluid-selection valve as a loop, wherein the oxygenation tubing comprises oxygen-permeable walls, and wherein the IVD analyzer further comprises a pump and a controller configured to control the pump and the fluid-selection valve for transporting deoxygenated calibration fluid from the fluid-supply unit into the oxygenation tubing, to wait a predetermined time required for oxygenation of the deoxygenated calibration fluid via oxygen uptake from ambient air through the tubing walls until the required level of oxygenation is obtained, and to transport the thereby obtained oxygenated calibration fluid into the sensor path for calibration of the at least one sensor.

2. The IVD analyzer according to claim 1, wherein the at least one sensor is a metabolite sensor including at least one of a glucose sensor and a lactate sensor.

3. The IVD analyzer according to claim 1, further comprising an oxygenation tubing for each different deoxygenated calibration fluid to be oxygenated.

4. The IVD analyzer according to claim 1, wherein the controller is further configured to control the pump and the fluid-selection valve to transport any other fluid from the fluid-supply unit into the sensor path while the at least one oxygenation tubing is fluidically isolated and the deoxygenated calibration fluid is being oxygenated.

5. The IVD analyzer according to claim 1, wherein the controller is further configured to control the pump and the fluid-selection valve to transport the oxygenated calibration fluid out of the oxygenation tubing into the sensor path while introducing fresh deoxygenated fluid into the oxygenation tubing to be oxygenated.

6. An automatic method of calibrating a sensor located in a flow-through sensor path of a detecting unit of an in-vitro diagnostic (IVD) analyzer involving a reaction with oxygen in a sample in order to determine a sample parameter and requiring at least one oxygenated calibration fluid with a certain level of oxygenation for calibration, the method comprising controlling by a controller a pump and a fluid-selection valve, including
transporting deoxygenated calibration fluid from a fluid-supply unit into an oxygenation tubing having two ends fluidically connected to the fluid-selection valve as a loop, the oxygenation tubing comprising oxygen-permeable walls, the fluid-selection valve comprising one or more fluid input ports for selecting at least one fluid at a time, and a common outlet port fluidically connected or connectable via a fluidic line to the sensor path,
waiting a predetermined time required for oxygenation of the deoxygenated calibration fluid via oxygen uptake from ambient air through the tubing walls until the required level of oxygenation is obtained, thereby obtaining an oxygenated calibration fluid, and
transporting the thereby obtained oxygenated calibration fluid into the sensor path and calibrating the at least one sensor.

7. The method according to claim 6, wherein the at least one sensor is a metabolite sensor including at least one of a glucose sensor and a lactate sensor.

8. The method according to claim 6, further comprising transporting different deoxygenated calibration fluids to be oxygenated into respective oxygenation tubings

9. The method according to claim 6, further comprising controlling the pump and the fluid-selection valve to transport any other fluid from the fluid-supply unit into the sensor path while the at least one oxygenation tubing is fluidically isolated and the deoxygenated calibration fluid is being oxygenated.

10. The method according to claim 6, further comprising controlling the pump and the fluid-selection valve to transport the oxygenated calibration fluid out of the oxygenation tubing into the sensor path while introducing fresh deoxygenated fluid into the oxygenation tubing to be oxygenated.
